(19) United States
(12) Patent Application Publication
(10) Pub. No.: US 2025/0378968 A1
(43) Pub. Date: Dec. 11, 2025

(54) Artificial Intelligence Healthcare Communication System

(71) Applicant: Healthcare Interactive, Inc., Glenwood, MD (US)

(72) Inventors: Henry H. Cha, Glenelg, MD (US); Xavier Cha, Glenwood, MD (US); Alex Straub, Glenwood, MD (US)

(21) Appl. No.: 19/230,026

(22) Filed: Jun. 5, 2025

Related U.S. Application Data
(60) Provisional application No. 63/656,590, filed on Jun. 5, 2024.

(51) Int. Cl. G16H 80/00; G16H 20/00; G16H 50/30

(52) U.S. Cl. CPC G16H 80/00; G16H 20/00; G16H 50/30

(57) ABSTRACT

An AI healthcare planner system including a processor and a computer readable medium having instructions for: proactively directing a first query to an incentive management expert resource to determine a number of points required for a healthcare recipient to reach a predetermined point total associated with an incentivized health goal; proactively directing a second query to a personality classification expert resource to determine a personality classification of the healthcare recipient; proactively directing a third query to a case management expert resource to identify health conditions of the healthcare recipient; proactively directing a fourth query to a care plan expert resource; and devising a plan of periodic communications to be sent to the healthcare recipient, based on a combination of information from the personality classification expert resource and the care plan expert resource, in order to advise the healthcare recipient with respect to their healthcare.

### CROSS-CITATION OF RELATED APPLICATIONS

This application claims priority to U.S. Provisional Application No. 63/656,590, filed Jun. 5, 2024, the entire disclosure of which is hereby incorporated by reference.

### TECHNICAL FIELD

The present disclosure generally relates to a healthcare communication system and, more particularly, to a healthcare communication system that utilizes artificial intelligence expert resources and configuration-driven architectures to automatically stage, build, and send personalized healthcare communications for advising and counseling healthcare recipients through comprehensive health insurance programs.

### BACKGROUND

Healthcare organizations, insurance companies, and other entities face significant challenges in effectively communicating with healthcare recipients regarding compliance requirements, wellness programs, and benefit utilization. Current manual approaches to healthcare communication suffer from numerous shortcomings that limit their effectiveness and scalability.

Traditional healthcare communication systems are severely limited in their capacity and personalization capabilities. Manual generation of healthcare communications is extremely slow, as an example with individual agents for case management with RN certified human capital, they are typically able to generate only 60-80 personalized communication plans per month. This low throughput necessitates large staffing requirements to serve even modest member populations, creating significant operational costs and scaling challenges. Even with increased staffing, the resulting communications suffer from quality issues, being too generic and insufficiently customized to individual healthcare recipients' specific needs, conditions, and circumstances.

Furthermore, healthcare organizations struggle with additional communication challenges including: coordinating, staging, building, and sending communications across large member populations; providing consistent, evidence-based health guidance while maintaining regulatory compliance; integrating multiple data sources including compliance status, incentive programs, and health risk assessments; delivering personalized content that adapts to individual personality classifications and health conditions; and managing the oversight and approval processes required for clinical accuracy and regulatory adherence.

The complexity of modern healthcare communication requires integration across multiple domains including compliance monitoring, incentive management, personality classification, and case management where a human resource will struggle compiling across too many data sources. Existing solutions typically address only one or two of these areas, creating gaps in service delivery and communication effectiveness.

Furthermore, traditional healthcare communication systems lack sophisticated feedback mechanisms and iterative improvement capabilities. Manual communication approaches do not effectively capture, analyze, or incorporate member feedback and sentiment data to improve subsequent communications. Without systematic feedback collection and analysis, healthcare organizations cannot optimize communication effectiveness, member engagement, or health outcomes over time. The absence of iterative learning mechanisms results in static communication strategies that fail to adapt to changing member needs, preferences, and health conditions, limiting the effectiveness of long-term care management programs.

There is a need in the art for a comprehensive AI-powered healthcare communication system that addresses these multiple challenges through an integrated approach capable of automated communication staging, building, and delivery while maintaining personalization, clinical accuracy, and regulatory compliance at scale.

### SUMMARY

The present disclosure is directed to a healthcare communication system that utilizes artificial intelligence expert resources and configuration-driven architectures to automatically stage, build, and send personalized healthcare communications for advising and counseling healthcare recipients. In addition, the system incorporates iterative feedback self-improvement mechanisms that continuously enhance communication effectiveness through sentiment analysis, member response evaluation, and adaptive content optimization. With the disclosed system, an entity can prepare thousands or more personalized communications per month due to the automation and systematic approach to communication generation, while continuously improving communication quality through feedback analysis.

The communication system operates through a three-phase process: staging communications based on eligibility criteria and scheduling parameters; building personalized content through AI expert consultation and sectional assembly; and sending interactive communications through multiple delivery channels while tracking engagement and compliance.

The system is enhanced by a supervisor application/tool, which enables licensed healthcare professionals to oversee and customize communication campaigns for large member populations while maintaining regulatory compliance and clinical accuracy

The system incorporates the supervisor application/tool functionality that enables licensed healthcare professionals to manage communication campaigns for thousands of healthcare recipients simultaneously through profile-based segmentation, clinical customization interfaces, and comprehensive regulatory compliance frameworks.

The system incorporates comprehensive feedback collection and analysis capabilities that enable iterative self-improvement through sentiment analysis of member responses, engagement metrics evaluation, and adaptive content optimization. The feedback system utilizes natural language processing and metric based algorithms to analyze member sentiment, satisfaction levels, and communication preferences from interactive feedback mechanisms embedded in communications. This feedback data is systematically processed to identify optimization opportunities, refine expert resource parameters, and enhance future communication generation for improved member engagement and health outcomes.

In one aspect, the present disclosure is directed to a healthcare communication system. The system includes a device processor and a non-transitory computer readable medium having stored thereon instructions, executable by the processor, for performing the following steps: accessing configuration-driven architecture that defines communication staging, building, and sending parameters; proactively directing queries to multiple expert resources including an incentive management expert resource, a personality classification expert resource, a case management expert resource, and a communication builder expert resource; processing healthcare recipient data through a sectional content generation pipeline that creates personalized and interactive healthcare communications; validating generated content through multiple AI expert validation systems; formatting communications using template-based systems with variable substitution; and delivering communications through multiple channels while maintaining comprehensive tracking and compliance monitoring.

In another aspect, the present disclosure is directed to a method of providing comprehensive healthcare communication services through the communication system. The method includes providing a controller with integrated AI expert resources; staging communications based on eligibility criteria, and scheduling configurations; building personalized content through expert consultation and sectional assembly; validating content for clinical accuracy and regulatory compliance; and sending communications through appropriate delivery channels while tracking member engagement and outcomes.

In another aspect, the present disclosure is directed to an artificial intelligence healthcare communication system, comprising: a device processor; and a non-transitory computer readable medium having stored thereon instructions, executable by the processor, for performing the following steps: proactively directing multiple queries to respective expert resources; devising a plan of periodic communications to be sent to the healthcare recipient, based on a combination of information from the queried expert resources, in order to advise the healthcare recipient with respect to their healthcare; collecting feedback data from healthcare recipients regarding the periodic communications; and integrating information related to the collected feedback data into the process of devising further plans of periodic communications.

Other systems, methods, features, and advantages of the disclosure will be, or will become, apparent to one of ordinary skill in the art upon examination of the following figures and detailed description. It is intended that all such additional systems, methods, features, and advantages be included within this description and this summary, be within the scope of the disclosure, and be protected by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like reference numerals designate corresponding parts throughout the different views.

FIG. 1 is a schematic diagram of an artificial intelligence healthcare communication system;

FIG. 2 is a flowchart illustrating a method of healthcare communication;

FIG. 3 is a schematic diagram of a configuration-driven communication pipeline;

FIG. 4 is a schematic diagram of the multi-expert validation and processing system;

FIG. 5 is a schematic diagram showing the comprehensive iterative feedback self-improvement system; and

FIG. 6 is an example of a personalized healthcare communication generated by the communication system.

### DETAILED DESCRIPTION

The present disclosure is directed to a healthcare communication system that utilizes artificial intelligence expert resources and configuration-driven architectures to automatically stage, build, and send personalized healthcare communications for advising and counseling healthcare recipients. These expert resources comprise advanced AI systems and databases organized and categorized to facilitate accurate retrieval and processing of healthcare information. The expert resources include data analysis modules configured to provide statistical data and personalized insights utilized for preparing comprehensive communication plans.

The disclosed artificial intelligence healthcare planner system may include a device processor and a non-transitory computer readable medium having stored thereon instructions executable by the processor for coordinating the staging, building, and sending of healthcare communications at scale.

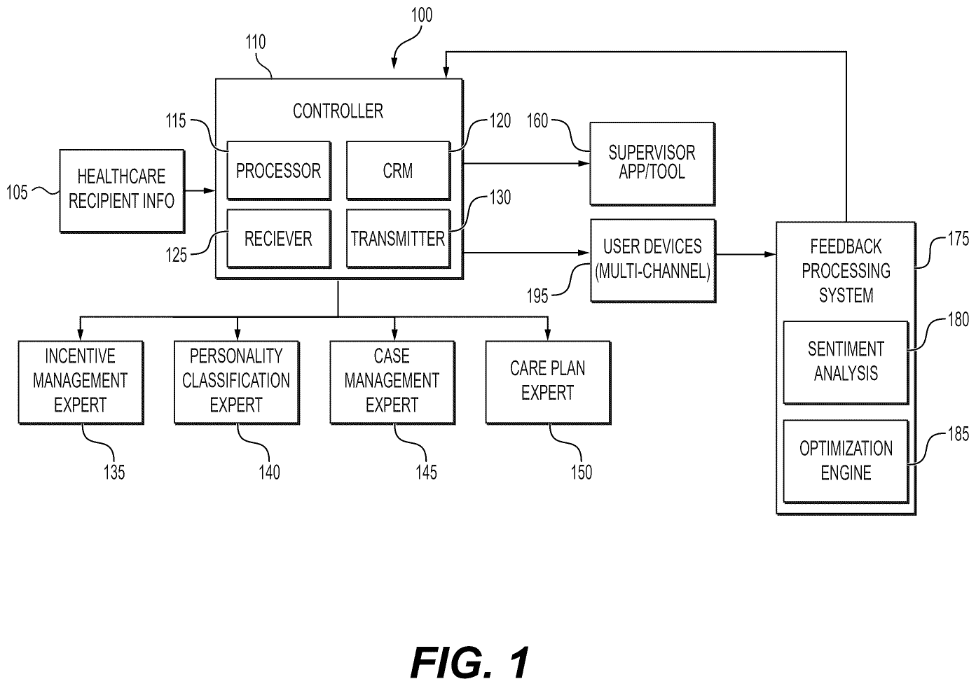

FIG. 1 is a schematic diagram of an artificial intelligence healthcare communication system. As shown in FIG. 1, an artificial intelligence healthcare communication system 100 is configured to receive healthcare recipient information (105), such as an identification of a healthcare recipient. The healthcare recipient information 105 may be received by a controller 110 that coordinates the staging, building, and sending phases of communication generation.

The controller 110 may include various computing and communications hardware. For example, controller 110 may include a device processor 115 and a non-transitory computer readable medium 120 including instructions executable by the device processor 115. The computer readable medium 120 may include any suitable computer readable medium, such as a memory, e.g., RAM, ROM, flash memory, or any other type of memory known in the art. The controller 110 may include other computing hardware, such as servers, integrated circuits, displays, and networking components.

The controller 110 includes networking hardware configured to interface with other network nodes, including local area networks (LAN), wireless local area networks (WLAN), and wide area networks. The controller 110 includes a receiver 125 and a transmitter 130, which may be combined in a transceiver in some embodiments. These components enable communication with healthcare information systems, member databases, and delivery channels through various formats including local network communications, internet communications, satellite communication, and radiofrequency signals.

The controller 110 may be provided at any suitable location, including healthcare organization headquarters, insurance carrier facilities, or dedicated cloud-based hosting environments configured to coordinate the communication staging, building, and sending processes securely and efficiently.

The instructions stored on computer readable medium 120 may be for proactively directing queries systematically to respective expert resources to retrieve information upon which the communication plans may be based. For example, the computer readable medium 120 may include instructions for proactively directing a first query to an incentive management expert resource 135 to determine compliance status, points earned, a number of points required for a healthcare recipient to reach a predetermined point total associated with reaching an incentivized health goal, and incentive eligibility for healthcare recipients. (It will be understood that, when discussing “expert resources” herein, for convenience the respective terms may be simply referred to as “experts.” For example, the “incentive management expert resource” may simply be referred to as the “incentive management expert.”) That is, among other things, the incentive management expert 135 may determine what incentives are in place for a healthcare recipient and what number of points are needed to satisfy the goal in order to receive the incentive (i.e., the reward, such as a financial incentive). Further, the incentive management expert resource 135 analyzes member participation in wellness programs, compliance activities, and reward structures to provide detailed insights into member progress and motivation factors.

In addition, the computer readable medium 120 may include instructions for proactively directing a second query to a personality classification expert resource 140 to determine a personality classification and communication preferences of healthcare recipients. Any statistical modeling may be utilized to determine the personality of the recipient. For example, in some embodiments, the personality classification expert resource 140 may utilize Latent Class Modeling to determine the personality of the healthcare recipient. Alternatively, or additionally, in some embodiments, the personality classification expert resource 140 may utilize Prochaska Readiness to Change (RtC) Methodology to determine how ready a healthcare recipient is to change their healthcare related behavior. Information from these models enables the system to tailor communication tone, content structure, and engagement strategies to individual member preferences and psychological profiles. Various other suitable statistical models may be used to classify the healthcare recipient's personality.

In addition, the computer readable medium 120 may include instructions for proactively directing a third query to a case management expert resource 145 to identify health conditions, risk factors, and care management needs of healthcare recipients. In some embodiments, the case management expert 145 may utilize predictive modeling to determine conditions and associated risk levels. This analysis incorporates clinical data, health risk assessments, and historical utilization patterns to provide comprehensive member health profiles. In some cases, the conditions may be identified by categorization and/or risk. For example, in some embodiments, the predictive modeling yields condition categories and the amount of risk there is in each condition.

The computer readable medium 120 may also include instructions for proactively directing a fourth query to a care plan expert resource 150 based on a combination of information retrieved from incentive management expert resource 135, personality classification expert resource 140, and case management expert resource 145. The care plan expert resource 150 is further configured to synthesize this multi-dimensional information to devise personalized communication strategies, including plan of periodic communications to be sent to the healthcare recipient in order to advise them with respect to their healthcare. In addition, care plan expert resource is configured to devise content recommendations tailored to individual member needs, preferences, and heath goals.

Further, controller 110 may be configured to send the periodic communications to the healthcare recipient. Accordingly, as shown in FIG. 1, controller 110 may be configured to communicate with a user device 155, such as a personal electronic device of the healthcare recipient. User device 155 may be any suitable electronic device that can receive communications. For instance, user device 155 may be a smart phone, tablet, laptop computer, or other computing device. System 100 may be configured to deliver communications via multiple channels. For example, controller 110 communicates with user devices 155, including personal electronic devices such as smartphones, tablets, laptop computers, and other computing devices capable of receiving healthcare communications through email, mobile applications, text messaging, and web portals.

As shown in FIG. 1, system 100 also integrates a supervisor application/tool 160 that enables licensed healthcare professionals to oversee and customize communication campaigns for large member populations. Supervisor application/tool 160 provides profile-based segmentation capabilities, clinical customization interfaces, and comprehensive regulatory compliance monitoring to ensure all communications meet professional standards and regulatory requirements.

It will be understood that the order in which the expert resources are consulted may vary. Further, in some embodiments, more or fewer expert resources may be consulted in retrieving information for the purpose of devising a communication plan for a healthcare recipient. In some cases, two or more expert resources may be consulted simultaneously. In other cases, two or more expert resources may be consulted in a predetermined sequence. Further, in some embodiments, upon consulting with a first expert resource, a determination of which expert resource should be consulted next may be based on the information retrieved from the first expert resource. In addition, the information retrieved from the various expert resources may be prioritized such that the information retrieved from certain expert resources is emphasized more than the information retrieved from certain other expert resources in terms of its effect on the communications plan.

In some embodiments, the care plan expert resource 150 is configured to create a schedule of communications to be sent to the healthcare recipient. In addition, the communications may be tailored based on the healthcare recipient's health conditions identified by the case management expert resource 145. In some embodiments, the care plan expert resource 150 is configured to create communications that suggest which incentives the healthcare recipient should focus on, based on the health care recipient's conditions identified by the case management expert resource 145, for the most impact on their health.

The devised communications may be topically categorized to include awareness and motivation, reinforcement and community building, and long-term engagement. These categories may be periodized. For example, communications regarding awareness and motivation may be sent during the first month. Reinforcement and community building communications may be sent during the second month. And long-term engagement communications may be sent during the third month. The time table and schedule of communications may vary and may depend on the conditions of the healthcare recipient and/or other information about the healthcare recipient.

In some embodiments, the awareness and motivation communications may include messages regarding personalized tips, interactive engagement, and a progress check. The reinforcement and community building communications may include messages regarding success stories, peer support, expert advice, and an incentive update. The long-term engagement communications may include messages regarding health benefits, new challenges, regular check-ins, and rewards and recognition.

The communication system operates through a systematic three-phase approach: 1.) staging communications based on eligibility criteria and scheduling parameters defined in configuration files; 2.) building personalized content through expert consultation and sectional assembly processes; and 3.) sending communications through appropriate delivery channels while maintaining comprehensive tracking and compliance monitoring.

In the staging phase, the system evaluates member eligibility based on configurable criteria including program enrollment, compliance status, communication preferences, and scheduling parameters. The staging process ensures appropriate timing and relevance of communications while respecting member preferences and regulatory requirements.

In the building phase, the system generates personalized communication content through systematic consultation of expert resources and sectional assembly of communication components. Each communication includes customized sections such as subject lines, welcome messages, compliance status updates, goals and rewards information, personalized wellness tips, and motivational conclusions.

In the sending phase, the system delivers communications through appropriate channels based on member preferences and tracks engagement, responses, and outcomes. The system maintains comprehensive logs for compliance monitoring and optimization of future communications.

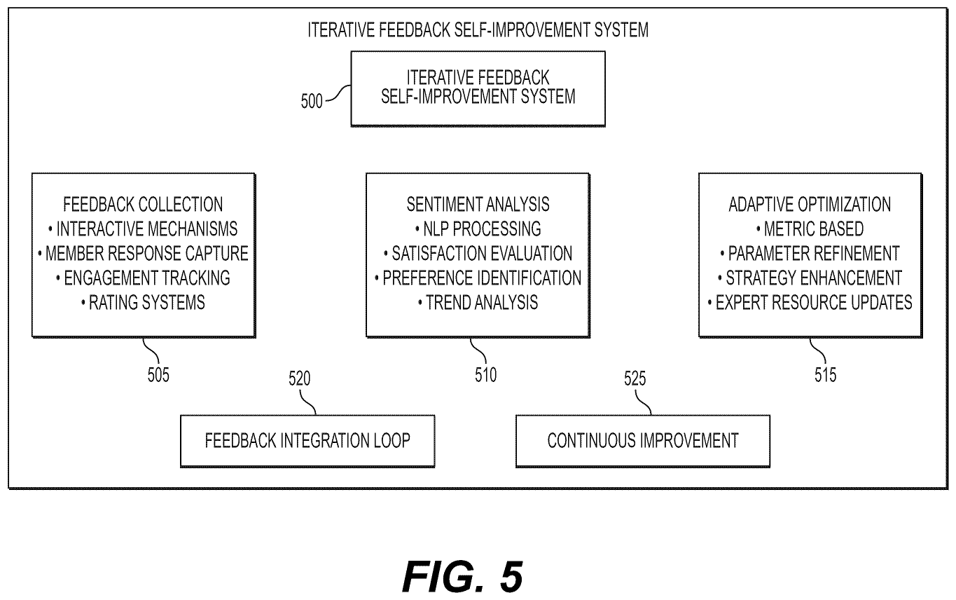

In addition, the communications system 100 incorporates comprehensive iterative feedback self-improvement capabilities that continuously enhance communication effectiveness through systematic collection, analysis, and integration of member feedback data. The iterative feedback self-improvement system (see system 500 in FIG. 5) utilizes advanced sentiment analysis, engagement metrics, and other statistical evaluations to optimize communication strategies and content generation processes based on real-world member responses and outcomes.

The feedback collection system (505) is integrated into all communications through interactive feedback mechanisms that enable members to provide responses, ratings, and comments directly within communications. Referring again to FIG. 1, a feedback processing system 175 includes mechanisms such as structured rating systems, open-text response fields, and engagement tracking that monitors member interactions with communication content. Feedback processing system 175 maintains comprehensive logs of all feedback data while ensuring HIPAA compliance and member privacy protection.

A sentiment analysis processing component 180, as shown in FIG. 1 and detailed in FIG. 5 as component 510, utilizes natural language processing algorithms to analyze member feedback text for sentiment indicators, satisfaction levels, and preference patterns. The system identifies positive and negative sentiment trends, communication preferences, and effectiveness indicators from member responses. This sentiment data is aggregated and analyzed to identify patterns across member populations, communication types, and content categories.

As also shown in FIG. 1, an adaptive optimization engine 185 (also detailed as component 515 in FIG. 5), processes feedback data to enhance expert resource parameters, content generation algorithms, and communication strategies. The system utilizes feedback techniques to identify successful communication patterns, optimize messaging tone and content structure, and refine personalization algorithms based on member response data. This continuous optimization process ensures that communication effectiveness improves over time through systematic learning from member interactions.

System 100 may further include a feedback integration system (see element 520 in FIG. 5), which coordinates with all expert resources to incorporate learned insights into future communication generation. The incentive management expert resource 135 utilizes feedback data to optimize motivation strategies. The personality classification expert resource 140 refines communication tone and structure based on member preferences. In addition, the care plan expert resource 150 adapts health guidance based on member engagement patterns. The continuous improvement process approach (see element 525 in FIG. 5) ensures that all aspects of communication generation benefit from continuous feedback-driven improvement.

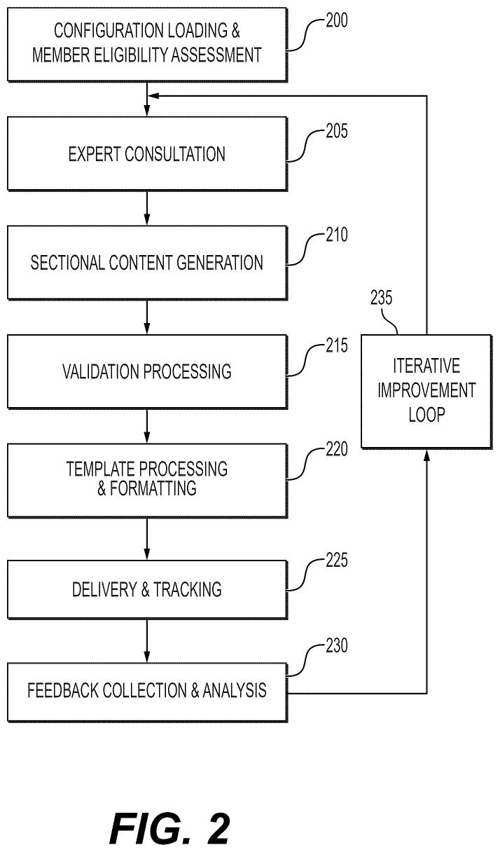

FIG. 2 is a flowchart illustrating a method of healthcare communication. The method begins with configuration loading and member eligibility assessment (step 200), where the system accesses JSON-based configuration files that define communication parameters, schedules, and criteria for member participation. Further, it illustrates the integration of feedback collection and analysis within the comprehensive communication process flow. The iterative improvement loop demonstrates how member feedback flows back into the system to enhance future communications, creating a continuous cycle of optimization that improves communication effectiveness over time. This feedback-driven approach enables the system to adapt to changing member needs, preferences, and health conditions while maintaining clinical accuracy and regulatory compliance.

The method includes a number of process steps executed by the controller. For example, the method includes systematic expert consultation (step 205), where queries are proactively directed by the controller to the incentive management expert resource, personality classification expert resource, case management expert resource, and care plan expert resource to gather comprehensive member insights and communication requirements.

The method includes sectional content generation (step 210), where personalized communication content is assembled through AI-powered generation of individual sections based on expert insights, member data, and configuration specifications.

In addition, the method includes comprehensive validation processing (step 215), where generated content undergoes multiple validation checks including medical accuracy verification, compliance assessment, and quality assurance review to ensure appropriate content delivery.

The method further includes template processing and formatting (step 220), where validated content is applied to appropriate communication templates with variable substitution and formatting for specific delivery channels.

The method includes delivery and engagement tracking (step 225), where communications are sent through designated channels and member engagement, responses, and outcomes are tracked for compliance monitoring and program optimization.

Finally, the method includes feedback collection and analysis (step 230), and the execution of an iterative improvement loop 235 by which the collected feedback is fed back into the process in order to improve the quality and usefulness of the communications derived by the method.

It may be appreciated that the steps of the various processes discussed above could be performed in different orders in some other embodiments.

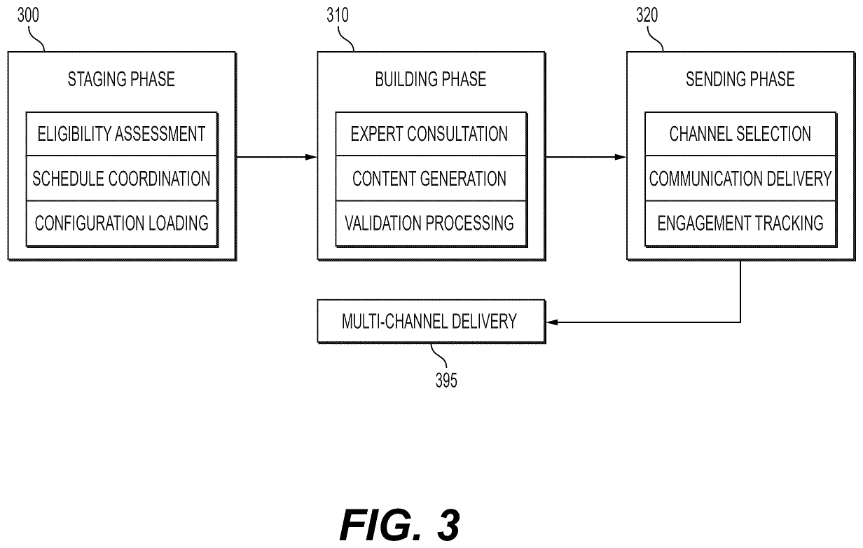

FIG. 3 is a schematic diagram of a configuration-driven communication pipeline. The pipeline demonstrates the systematic approach to communication generation through configurable parameters and expert resource coordination.

As shown in FIG. 3, the process of generating communications includes a staging phase 300, which includes eligibility assessment, scheduling coordination, and configuration loading to determine which members receive communications and when. The process further includes a building phase 310, which includes expert consultation, content generation, and validation processes to create personalized communications. Next, the process includes a sending phase 320, which includes delivery channel selection, communication delivery, and engagement tracking. Once the process of generating communications is completed, the system executes multi-channel delivery (step 395), which expands upon delivery channel selection to be able to send communications through different mediums (e.g., text, email, mobile app messaging, SMS, etc.). Further, the system may include a web portal with comprehensive engagement tracking.

As an example of the types of communications generated, the system may derive a plan of communications that includes various topics for communications at various times. As an example, during the first month of the plan, communications may be directed generally to “Awareness and Motivation.” More specifically, Month 1 may include communications for the first four weeks that are respectively directed to topics such as “Introduction,” “Personalized Tips,” “Interactive Engagement,” and “Progress Check.”

Continuing with this example, Month 2 communications may be directed generally to “Reinforcement & Community Building.” More specifically, Month 2 may include communications for the next four weeks that are respectively directed to topics such as “Success Stories,” “Peer Support,” “Expert Advice,” and “Incentive Update.”

The weekly topics for Month 3 communications may be directed generally to “Long-Term Engagement.” More specifically, Month 3 may include communications for the next four weeks that are respectively directed to topics such as “Health Benefits,” “New Challenges,” “Regular Check-Ins,” and “Reward and Recognition.”

It will be understood that the topics may vary from plan to plan, recipient to recipient, etc. For example, the topics may vary depending on the level of severity of the health conditions of the healthcare recipient. The topics may also vary depending on the personality of the patient, and the patient's willingness/readiness to change as discussed above.

The system may include a supervisor application/tool, which enables licensed healthcare professionals to manage communication campaigns for thousands of healthcare recipients through profile-based segmentation systems, clinical customization interfaces, and comprehensive regulatory compliance frameworks. This supervisory capability transforms traditional one-to-one care management into scalable, technology-enhanced communication management while maintaining clinical excellence and regulatory adherence.

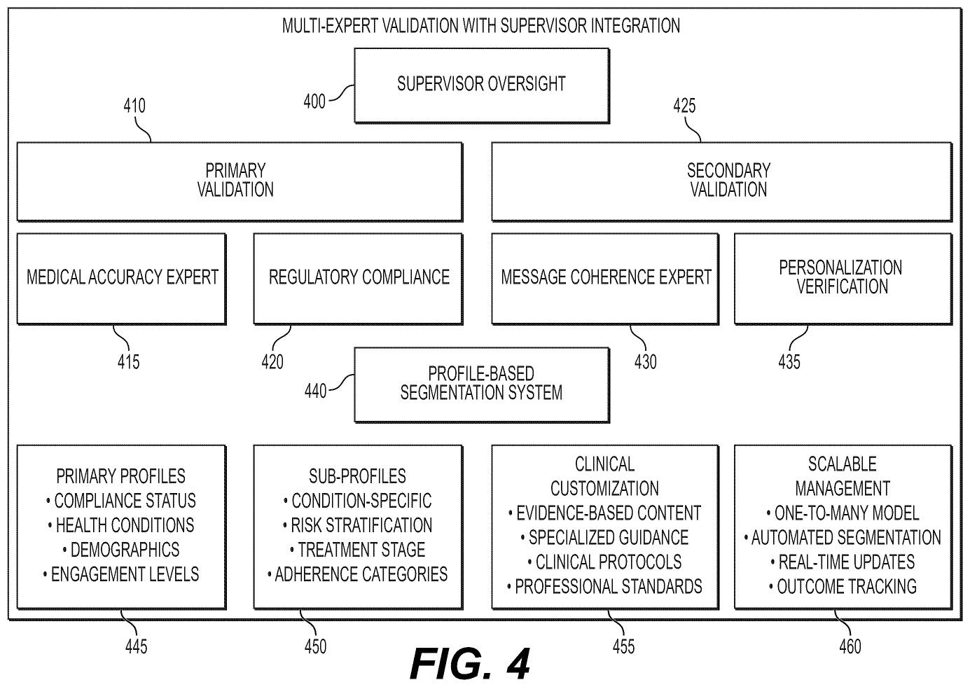

FIG. 4 is a schematic diagram of a multi-expert validation and processing system. The supervisor application/tool 400 may implement a multi-step validation process. For example, as illustrated in FIG. 4, supervisor app/tool 400 enables licensed healthcare professionals to manage communication campaigns through profile-based segmentation system (440) with primary profiles (445) and sub-profiles (450), clinical customization (455), regulatory compliance (420), and scalable management (460), while ensuring quality through integrated validation systems (410) and (425).

The primary validation (410) is an assessment of clinical accuracy as well as a compliance verification. The process of primary validation (410) involves the consultation of a medical accuracy expert resource 415, including a reviews of health content and clinical guideline compliance. This is an evidence-based validation.

The primary validation (410) also includes an assessment of regulatory compliance (420), which involves a review of HIPAA compliance, state and/or federal regulations, and professional standards.

The secondary validation (425) is an assessment of quality assurance and personalization. The process of secondary validation (425) involves consultation of a message coherence expert resource (430), which includes logical flow verification, a readability assessment, and engagement optimization. The secondary validation (425) also includes personalization verification (435), which involves individual customization, profile-based adaptation, and preference alignment.

In addition, the profile-based segmentation system (440) provides hierarchical member categorization for scalable communication management. The profile-based segmentation system (440) relies on primary profiles (445) for such things as compliance status, health conditions, demographics, and engagement levels. In addition, the profile-based segmentation system (440) relies on sub-profiles (450) for such things as condition-specific information, risk stratification, treatment stage information, and adherence categories.

Also, the profile-based segmentation system (440) provides clinical customization (455), relying on evidence-based content, specialized guidance, clinical protocols, and professional standards. Further, the profile-based segmentation system (440) provides scalable management (460) based on a one-to-many model, automation segmentation, real-time updates, and outcome tracking.

As noted with respect to FIG. 1 above, the system may include an iterative feedback self-improvement system to enhance the communication plans over time. FIG. 5 is a schematic diagram showing the comprehensive iterative feedback self-improvement system. As shown in FIG. 5, an iterative feedback self-improvement system 500 may be provided for continuous enhancement through member response analysis and metric based optimization.

System 500 is configured for feedback collection (505), which involves interactive mechanisms, member response capture, engagement tracking, and rating systems. System 500 is also configured for sentiment analysis (510), which involves natural language processing (NLP), satisfaction evaluation, preference identification, and trend analysis. Further, system 500 is configured for adaptive optimization (515), which is metric based and involves parameter refinement and strategy enhancement, while receiving expert resource updates.

As shown in FIG. 5, system 500 implements a feedback integration loop (520), which involves the systematic incorporation of learned insights into expert resource parameters and communication generation processes. Further, system 500 implements continuous improvement (525) via the iterative enhancement of communication effectiveness through data-driven optimization and adaptive learning mechanisms.

The expert resources may be consulted in various sequences and combinations depending on configuration specifications and member characteristics. In some embodiments, expert resources are consulted simultaneously to optimize processing efficiency. In other embodiments, expert resources are consulted sequentially with prioritization based on member risk levels or communication urgency.

The care plan expert resource 150 (see FIG. 1) is configured to create comprehensive schedules of communications customized to individual member needs and delivered over extended periods. Communications are tailored based on healthcare recipients' health conditions, compliance status, personality classifications, and engagement preferences identified through expert resource analysis.

The disclosed communication system creates communications that provide personalized guidance on compliance activities, wellness programs, and health management strategies based on individual member profiles, expert resource insights, and iterative feedback analysis. The system suggests specific actions and focuses areas based on member conditions, compliance status, incentive opportunities, and learned preferences from prior communication feedback for maximum impact on health outcomes and member satisfaction.

Communications generated by the disclosed communication system are topically categorized and periodized to provide systematic engagement over time. Categories include compliance guidance, wellness education, incentive updates, and motivational support. The timing and content of communications are customized based on member characteristics, program requirements, and engagement history.

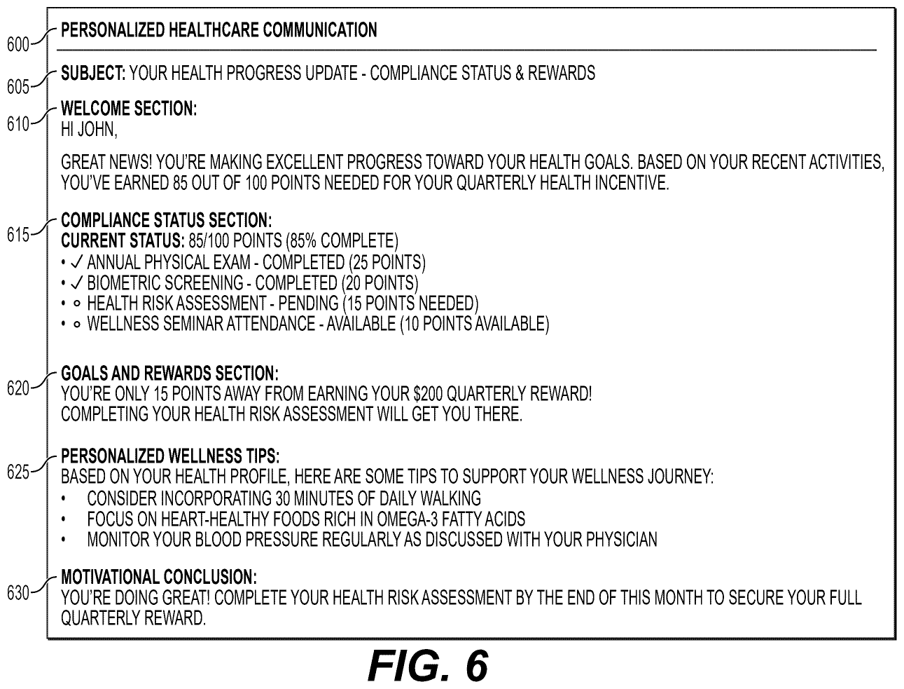

FIG. 6 is an example of a personalized healthcare communication generated by the communication system. As shown in FIG. 6, the communication 600 may be structured as a traditional letter or email. For example, the communication may have a personalized subject line 605 tailored to the member's current compliance status and program participation, reviewed and approved through the supervisor app/tool.

In addition, the message may include a welcome section 610, which provides a personalized greeting and status update based on the member's current compliance state, achievement level, and engagement history under professional supervision.

Further, the message may have a compliance status section 615, which includes specific information about the member's current standing and required actions, which are presented in an encouraging and supportive manner with professional approval.

Also, a goals and rewards section 620 of the message highlights relevant incentives and achievements based on the member's program participation and progress under professional oversight.

A personalized wellness tips section 625 provides health guidance tailored to the member's conditions and goals while avoiding specific medical condition naming for HIPAA compliance, with all health recommendations reviewed and approved by healthcare professionals through the supervisor app/tool.

Finally, a motivational conclusion 630 provides encouragement and next steps customized to the member's personality classification and engagement preferences under professional supervision.

While various embodiments have been described, the description is intended to be exemplary, rather than limiting, and it will be apparent to those of ordinary skill in the art that many more embodiments and implementations are possible that are within the scope of the embodiments. Although many possible combinations of features are shown in the accompanying figures and discussed in this detailed description, many other combinations of the disclosed features are possible. Any feature of any embodiment may be used in combination with or substituted for any other feature or element in any other embodiment unless specifically restricted. Therefore, it will be understood that any of the features shown and/or discussed in the present disclosure may be implemented together in any suitable combination. Accordingly, the embodiments are not to be restricted except in light of the attached claims and their equivalents. Also, various modifications and changes may be made within the scope of the attached claims.

We claim:

1. An artificial intelligence healthcare communication system, comprising:
a device processor; and
a non-transitory computer readable medium having stored thereon instructions, executable by the processor, for performing the following steps:
proactively directing a first query to an incentive management expert resource to determine a number of points required for a healthcare recipient to reach a predetermined point total associated with reaching an incentivized health goal;
proactively directing a second query to a personality classification expert resource to determine a personality classification of the healthcare recipient;
proactively directing a third query to a case management expert resource to identify health conditions of the healthcare recipient;
proactively directing a fourth query to a care plan expert resource based on a combination of information retrieved from the incentive management expert resource and the case management expert resource; and
devising a plan of periodic communications to be sent to the healthcare recipient, based on a combination of information from the personality classification expert resource and the care plan expert resource, in order to advise the healthcare recipient with respect to their healthcare.

2. The system of claim 1, wherein the incentivized health goal is associated with at least one of the following: compliance programs, wellness activities, and financial incentives.

3. The system of claim 1, wherein the personality classification expert resource utilizes Latent Class Modeling to determine the personality of the healthcare recipient.

4. The system of claim 1, wherein the personality classification expert resource utilizes Prochaska Readiness to Change (RtC) Methodology to determine how ready a healthcare recipient is to change their healthcare behavior.

5. The system of claim 1, wherein the health conditions are identified by the case management expert resource using predictive modeling.

6. The system of claim 5, wherein the predictive modeling yields condition categories and the amount of risk there is in each condition.

7. The system of claim 1, wherein the care plan expert resource is configured to create a schedule of communications to be sent to the healthcare recipient.

8. The system of claim 7, wherein the communications are tailored based on the healthcare recipient's health conditions identified by the case management expert resource.

9. The system of claim 7, wherein the care plan expert resource is configured to create communications that suggest which incentives the healthcare recipient should focus on, based on the healthcare recipient's conditions identified by the case management expert resource, for the most impact on their health.

10. The system of claim 7, wherein the communications may be topically categorized to include awareness and motivation, reinforcement and community building, and long-term engagement.

11. The system of claim 10, wherein the awareness and motivation communications include messages regarding personalized tips, interactive engagement, and a progress check.

12. The system of claim 10, wherein the reinforcement and community building communications include messages regarding success stories, peer support, expert advice, and an incentive update.

13. The system of claim 10, wherein the long-term engagement communications include messages regarding health benefits, new challenges, regular check-ins, and rewards and recognition.

14. The system of claim 1, wherein the expert resources are consulted in a predetermined sequence.

15. The system of claim 1, wherein certain expert resources are consulted in response to information retrieved from certain other expert resources.

16. The system of claim 1, wherein the information retrieved from the expert resources is prioritized such that the information retrieved from certain expert resources is emphasized more than the information retrieved from certain other expert resources in terms of its effect on the communications.

17. The system of claim 1, further including a supervisor tool configured to facilitate oversight by healthcare professionals with respect to the generation of communication campaigns.

18. The system of claim 1, wherein the computer readable medium further includes instructions for delivering communications through multiple channels while maintaining comprehensive tracking and compliance monitoring.

19. A method of formulating a healthcare plan, comprising:
providing a controller including a device processor and a non-transitory computer readable medium having stored thereon instructions, executable by the processor;
proactively directing, with the controller, a first query to an incentive management expert resource to determine a number of points required for a healthcare recipient to reach a predetermined point total associated with reaching an incentivized health goal;
proactively directing, with the controller, a second query to a personality classification expert resource to determine a personality classification of the healthcare recipient;
proactively directing, with the controller, a third query to a case management expert resource to identify health conditions of the healthcare recipient;
proactively directing, with the controller, a fourth query to a care plan expert resource based on a combination of information retrieved from the incentive management expert resource and the case management expert resource; and
devising a plan of periodic communications to be sent to the healthcare recipient, based on a combination of information from the personality classification expert resource and the care plan expert resource, in order to advise the healthcare recipient with respect to their healthcare.

20. The method of claim 19, wherein the personality classification expert resource utilizes Latent Class Modeling to determine the personality of the healthcare recipient.

21. The method of claim 19, wherein the personality classification expert resource utilizes Prochaska Readiness to Change (RtC) Methodology to determine how ready a healthcare recipient is to change their healthcare behavior.

22. The method of claim 19, wherein the health conditions are identified by the case management expert resource using predictive modeling.

23. An artificial intelligence healthcare communication system, comprising:
a device processor; and
a non-transitory computer readable medium having stored thereon instructions, executable by the processor, for performing the following steps:
proactively directing multiple queries to respective expert resources;
devising a plan of periodic communications to be sent to the healthcare recipient, based on a combination of information from the queried expert resources, in order to advise the healthcare recipient with respect to their healthcare;
collecting feedback data from healthcare recipients regarding the periodic communications; and
integrating information related to the collected feedback data into the process of devising further plans of periodic communications.

24. The system of claim 23, wherein the periodic communications include feedback mechanisms embedded therein that are configured to facilitate the provision of feedback by the recipients of the periodic communications.

25. The system of claim 23, wherein the computer readable medium further includes instructions for utilizing sentiment analysis using natural language processing to analyze member feedback.

26. The system of claim 23, wherein integrating the information into the process of devising further plans of periodic communications includes integrating learned insights across multiple expert resources consulted as part of the process.

27. The system of claim 23, further including a supervisor tool configured to facilitate oversight by healthcare professionals with respect to the generation of communication campaigns.
